(19) United States
(12) Patent Application Publication
(10) Pub. No.: US 2024/0361324 A1
(43) Pub. Date: Oct. 31, 2024

(54) POINT OF SERVICE METHOD OF DETECTING, DIAGNOSING AND FOLLOWING CANCER AND OTHER PROLIFERATIVE CONDITIONS

(71) Applicants: Ahmed Fadiel-Metwaly, Smyrna, TN (US); Frederick Naftolin, Woodbridge, CT (US)

(72) Inventors: Ahmed Fadiel-Metwaly, Smyrna, TN (US); Frederick Naftolin, Woodbridge, CT (US)

(21) Appl. No.: 18/586,774

(22) Filed: Feb. 26, 2024

Related U.S. Application Data
(63) Continuation-in-part of application No. 17/864,713, filed on Jul. 14, 2022.
(60) Provisional application No. 63/221,742, filed on Jul. 14, 2021.

(51) Int. Cl. G01N 33/574; G01N 21/59; G01N 33/543; G01N 33/58

(52) U.S. Cl. CPC G01N 33/57449; G01N 21/59; G01N 33/543; G01N 33/581; G01N 2333/7151; G01N 2470/06

(57) ABSTRACT

p80 is a cancer/proliferation-related protein identified as being present in bodily fluids or tissues (including blood) of humans or animals afflicted with pre-malignant, malignant cells/tissues or proliferative conditions. The methods of the present disclosure provide a new diagnostic marker for screening of cancers and proliferative conditions. The present application discloses screening, diagnosis, and monitoring of these conditions by novel Point-of-Service tests. The addition of p80-bound tubulin testing is also claimed. Additionally, the present application discloses targeted treatment of cancer and proliferative conditions utilizing p80 binding agents, via a variety of delivery vehicles such as nanoparticles. The claims apply to application in domestic or wild animals.

### CROSS REFERENCE TO RELATED APPLICATION

This application claims benefit of the filing date of U.S. patent application Ser. No. 17/864,713, entitled, “USES AND APPLICATIONS IN CANCER AND OTHER PROLIFERATIVE CONDITIONS OF AGENTS INTERACTING WITH THE UNIQUE MICROTUBULE ASSEMBLY PROTEIN p80 OR ABNORMAL MEMBERS OF THE ERM FAMILY OF PROTEINS,” filed on Jul. 14, 2022, which claims benefit of the filing date of U.S. Provisional Patent Application Ser. No. 63/221,742, filed on Jul. 14, 2021. The teachings of the referenced application are herein incorporated by reference.

### INCORPORATION BY REFERENCE OF SEQUENCE LISTING

The Sequence Listing in an XML format, named as 42214_SequenceListing of 4 KB, created on Jul. 11, 2024, and submitted to the United States Patent and Trademark Office via Patent Center, is incorporated herein by reference.

### FIELD OF THE DISCLOSURE

The present disclosure relates to patient screening, diagnosis, treatment and follow up of cancer, neoplasms, and other proliferative conditions involving a novel oncoprotein, p80. Point of Service testing for screening and follow up of these lesions is included. For descriptive purposes, examples based on gynecologic cancers are depicted; however, these are only examples rather than limits of the use of these agents on developing or actual human and animal cancers, metastatic cells, and cells as are found during embryonic development or proliferative diseases. All pre-malignant, malignant, developing and proliferative disease cells, their contents and fragments are targeted in this disclosure.

### BACKGROUND OF THE DISCLOSURE

### The Problem Of Cancer, Proliferative Conditions and Diseases

Screening and diagnosis-Cancer is the second leading cause of death in the United States. Half of all men and one-third of all women in the U.S. will develop cancer. Today, millions of people are living with cancer or have had cancer. The sooner a cancer is found, and treatment begins, the better the chances for survival. However, the pre-malignant and frankly malignant cells may escape early diagnosis, grow, and metastasize, at which point the treatment becomes more difficult by order of magnitude. Although diagnosis and treatment have improved, there is an urgent need for early diagnosis, improved clinical staging and safe, efficacious treatment. The same applies to proliferative diseases such as blood cell cancers, “benign” brain tumors that kill by displacing normal cells and such diseases as fibromatosis and psoriasis. To illustrate, ovarian cancer is a preventable cancer in the ovaries' cells. The most common type of cancer arising from the ovary is ovarian epithelial cancer (OVCA) which may arise as a proliferative stage that leads to localized, “in situ” cancer and then invasive ovarian cancer or be the result of droplet seeding from fallopian tube cancer. A second type of OVCA arises from the fallopian tube and implants in the nearby ovary. A less common intraperitoneal cancer (idiopathic peritoneal cancer) apparently arises from the same peritoneum as covers the ovary and has a clinical course indistinguishable from OVCA. A fourth type is metastatic, from other organs' cancers, e.g., from breast cancer or stomach cancer.

Even in this era of increased interest in the health needs of women, OVCA stands out as a deadly disease in particular need of attention. Because of the lack of effective screening for OVCA, 2/3 of OVCA cases are metastatic at the time of detection and will not be cured. According to the American Cancer Society, ovarian cancer accounts for only 4% of all cancers among women but ranks fifth as a cause of their deaths from cancer. Over 14,000 women die each year from OVCA/PPC alone (2.3% of all cancer deaths), making it the leading cause of death from gynecologic malignancy. OVCA malignant cells migrating along adjacent peritoneum can progress extensively before becoming symptomatic locally or their metastases cause symptoms such as intraperitoneal fluid (ascites).

Pelvic examination or imaging is not successful in finding primary OVCA. Present markers, such as CA125, are non-specific and have many false positives. The result of these failures is that OVCA is usually detected at an advanced stage and the outcome is almost uniformly fatal. No dependable method of discovery of OVCA is available and no curative drug treatment exists for metastatic OVCA. Despite the availability of new therapies, the mortality rate attributed to OVCA has not changed significantly in the last 50 years. One result of this situation is the increased performance of prophylactic ovariectomy. This ablation of the ovary, and often the uterus and fallopian tubes, renders women infertile. It also may result in early death from cardiovascular disease.

Endometrial adenocarcinoma (ENDOCA) is the most common gynecological cancer in women. It arises from the endometrial glandular lining, and, unlike OVCA, it may metastasize by invading lymphatics and blood vessels to spread widely, as well as invading locally. ENDOCA begins as a proliferative condition, endometrial hyperplasia, that undergoes malignant degeneration. The treatment of persistent hyperplasia often requires hysterectomy, resulting in infertility. Although ENDOCA cells are of a similar level of aggressivity as OVCA, ENDOCA is not as lethal because of early diagnosis triggered by vaginal bleeding that it causes uterine bleeding or an abnormal ultrasound examination that sets off a diagnostic workup that exposes the ENDOCA and results in treatment before metastases occur. However, there is no simple or painless or certain method of performing this sampling. Usually, an endometrial biopsy or curettage is required, and thus repeated/screening testing is not feasible in large populations. Even then, all forms of endometrial biopsy have a low but definite rate of false negatives.

Cervical cancer (CXCA) arises from the epithelium of the uterine cervix, generally at the juncture of the squamous and columnar glandular epithelium. CXCA is almost always associated with human papillomavirus (HPV) infection and passes through a pre-invasive, proliferative stage. Early diagnosis requires sampling of the squamo-columnar junction. Although Pap smears have reduced the death rate of CXCA it has remained a major cause of women's deaths. Similarly, testing for the presence of HPV is not a test for cancer; rather, it is only a test for the predilection to cancer. Colposcopy is diagnostic, not suitable for general screening for CXCA.

Thus, OVCA, ENDOCA and CXCA are examples of preventable cancers which pass through proliferative pre-malignant stages before they achieve symptoms to trigger detection by present methods of diagnosis. However, by then it is often too late for OVCA treatment and usually results in hysterectomy and infertility in the case of ENDOCA and CXCA. There is a need to develop new cancer screening, diagnostic and treatment methods, particularly those for screening, diagnosing and/or inhibiting cancer before the invasion and/or metastasis. Additionally, even after then, metastatic cell diagnosis, localization and treatment are lacking.

Clinical Staging and targeted treatment-The treatment of OVCA, ENDOCA and CXCA are governed by the ability to properly define the extent of the lesion and location of metastases. Currently, there are no tumor-specific agents to allow either external (x-ray/MRI/other imaging) identification of their proliferating malignant cells or to assist in the clinical staging at surgery or to assess the completeness of removal techniques (surgery/focused ultrasound, etc.). The planning and execution of treatment is hindered by the lack of specific agents that can be used to sample biological fluids or be incorporated in imaging techniques to assess the effect of treatment or the possibility of recurrence. Further, the lack of specific agents targeting molecules specific to cancer and other proliferative lesions has hindered the development of agents that safely and selectively mark and aim at these lesions. Similarly, in the absence of cancer-specific molecules there are no specific non-sampling means of assessing cancer treatment effects.

These gynecological cancers are only examples of the cancers that strike women, men, children and animals. These descriptions are generic examples of the failure of present methods for screening, diagnostic testing and assessing treatment effects.

PROLIFERATIVE CONDITIONS: The microtubules and actin cytoskeleton are critical for cell division, intracellular metabolism and cell shape and motion during embryonic development and post-natal life. Errors in microtubule function or connection with the actin cytoskeleton will result in abnormalities in each of these functions that are characterized as abnormal proliferation and bizarre cellular morphology and cell dysfunction. While these may be detected by cell counts or cell morphology studies on blood cells, other lesions are usually accessible for direct testing and the need for screening tests is absolute. At present, the main screening by imaging for the presence of these diseases relies on by non-specific imaging techniques which may be replaced by the specific targeting embodied in this application.

LACK OF POINT-OF-SERVICE (POS) TESTING: Screening for cancer, or proliferative diseases should be available in all medical settings, should return results during the same encounter and should allow on-the-spot interpretation for the patient. While such testing is available for metabolic diseases, this is not the case for cancer screening. Pap smears are an example of the lack of these characteristics. Usually, the vaginal cytology, etc. must be sent to an off-site laboratory for specialist evaluation, or the patient must be sent to an off-site specialist for colposcopy etc. The patient must return to receive the results and counseling or receive a report in a form that may not allow appropriate interaction with the caregiver/doctor. Besides being wasteful of the patient's and doctor's time and resources, this is often psychologically challenging to the patient. On the other hand, POS testing is immediate and allows the patient to be immediately counseled and/or treated. This encourages more screening and earlier diagnoses.

### Microtubule-Associated Proteins, Cancer and Other Proliferative Diseases

EVIDENCE OF CANCER COMPONENTS IN BIOLOGICAL FLUIDS: Until recently there has been a lack of evidence for cancer, precancer or mutagenic molecules in biological fluids. Available tests such as CEA¹²⁵ are not specific for cancer.

It has now been shown that living and dead cancer cells regularly shed and absorb portions of their cells (cell fragments, micro-vesicles, exosomes, DNA, other molecules, etc.) that are found in intercellular spaces and biological fluids. These structures carry molecules that could identify/quantitate the presence of cancer and other proliferative disease cells. As well, molecules that bind to cancer or proliferative disease cells/molecules could be used to carry cargos of specific marking or treatment agents. FIGS. 12 illustrates the presence of p80 in sera from patients with ovarian cancer.

Microtubule (MT) function and disease-MT are constructed from segments of tubulin that are constantly being added to one end and removed from the other end. This is facilitated by microtubule-associated proteins (MAP's). The MT bind to the cytoskeletal network of actin fibers that are themselves bound to the cell membrane. In this manner the coordinated shortening of one end and elongation of the other end of the microtubule regulates cell motility, the separation of chromosomes during cell division, the furnishing of cellular components to regions of the cell and other vital functions and the development of cellular processes that are critical to intercellular movement such as occurs during metastasizing.

### SUMMARY OF THE DISCLOSURE

The disclosure is partly based on the discovery of a protein named p80 in cancers such as OVCA, ENDOCA, CXCA and all other non-reproductive cancers thus far tested. p80 which is a truncated MAP, has been discovered, isolated and sequenced. The truncated MAP, p80, contains the tubulin binding site that allows it to play a role in microtubule formation and dissolution but lacks the actin binding site common to all other MAPS and therefore cannot link the MT's to the actin cytoskeleton. In this manner, p80 occupies the tubulin binding site while blocking normal MT function. This is associated with cell de-differentiation and malignant cytology like that seen in stem cells. It is also shown p80 to be expressed in mesenchymal stem cells that exhibit similar cytological and proliferative characteristics as do cancer cells.

P80 is present in embryonic stem cells and in dedifferentiating ovarian epithelial cells as they undergo neoplastic changes in vitro. p80 may be present in proliferative lesions including blood cancers, fibromatoses, psoriasis, meningiomas, but is not expressed by normal cells in humans and animals. Other proliferative conditions include, but are not limited to, rheumatoid arthritis, atherosclerosis, idiopathic pulmonary fibrosis, scleroderma, cirrhosis of the liver, neovascularization or any other condition having a proliferation of cells. p80 may be a truncated form of the MAP1a or other MAP's and may compete for tubulin binding without associated actin binding, thus leaving untethered microtubules to fail in regulating mitosis and cell cytology because p80 lacks an actin-binding site, thereby interfering with the action of other MAP's and resulting in abnormalities of cell division, cell metabolism and cell shape/motility.

Since p80 is not expressed in normal, differentiated cells, specific p80-binding agents such as antibodies or small molecules may be used in humans and animals for screening for-and monitoring of cancer and other proliferative conditions; staging and clinical management or cancer and other proliferative conditions; cancer-targeting treatment and prevention of normal cells undergoing malignant transformation, pre-invasive (in situ) cancers, invasive cancers and other proliferative conditions. When utilized on a platform that allows immediate recognition of the p80 molecule by a specific antibody, the testing can be used to furnish a Point-of-Service test for p80 in biological fluids or in digests of surface cells such as are obtained during vaginal scrapping (pap smears), nipple aspiration, etc. In addition to its value as a cancer test, the ability to furnish a Point of Service test and immediate results is notable for its medical economic and patient care aspects.

The same measurements can be used to monitor the effects of treatment of cancers or other proliferative diseases that express p80. The same is true for discovery of recurrent disease.

The present disclosure relates to cancer tagging during the search for metastases, to assist in clinical staging and direct imaging of lesion size, etc., to monitor the effects of surgical or medical treatment. For these uses binding agents tagged with imaging-dense, chemiluminescent, fluorescing or radioactive materials may be used to detect cancers or other proliferating disease cells and masses by imaging or methods that could be applied during surgical staging of disease that depends on the presence, amount and location of metastases.

The present disclosure also relates to cancer targeting treatment—nanoparticles such as exosomes may be bound to glycocalyx antigens of cancer cells and then be internalized by endocytosis or enter by fusion with cell membranes. Once in the cells, after which they may be released from the vesicles and bind to p80-target proteins via outer nanoparticle- or p80 specific monoclonal antibodies. Thereafter, therapeutic agent cargos of the nanoparticles including chemotherapeutic drugs, microRNA's, toxic small molecules and radioactive isotopes would disable the p80 expressing cancer cells, but not cells that do not express p80. These actions depend on the presence of the target p80 protein; therefore, they should not affect normal cells.

The present disclosure also relates to cancer prevention—(1) p80 expressed in apparently normal cells or tissues or cancerous tissues or those of proliferating diseases may be sign of cryptic malignant degeneration and or proliferative changes and may serve as a target for treatment-laden p80-binding molecules and biomaterials that could forestall carcinogenesis. or (2) the expression of p80 could be causing neoplastic changes. Therefore, surgical or medical treatment could forestall the development of cancer.

Methods of the present disclosure can be used to detect and treat a variety of proliferative conditions, including solid cancers, blood cancers, sarcomas and (non-cancer) proliferative disorders.

The cancers and abnormally developing cells such as atypical hyperplasia's, hamartomas, fibromas that can be detected or treated using the subject method include, but are not limited to ovarian cancer, endometrial cancer, breast cancer, glioblastoma, schwannoma, meningioma, malignant mesothelioma, neurofibromatosis, colon cancer, oral cancer, or cancer selected from the group consisting of: lung cancer, prostate cancer, pancreatic cancer, leukemia, liver cancer, stomach cancer, uterine cancer, testicular cancer, brain cancer, non-Hodgkin's lymphoma, Hodgkin's lymphoma, Ewing's sarcoma, osteosarcoma, neuroblastoma, rhabdomyosarcoma, melanoma, and metastatic tumors of the brain or other organs.

One aspect of the present disclosure is directed to a method of screening, diagnosis, clinical staging or assessment of treatment for cancer or proliferative disease in a subject in need thereof, the method comprising: (a) obtaining a tissue/cellular or biological fluid sample from the subject; (b) diluting the tissue sample in a lysing buffer solution; (c) contacting the tissue sample with a monospecific antibody that recognizes p80 protein; (d) detecting complex formation or binding of the antibody to p80 protein; (e) wherein complex formation or binding is detected with an ELISA colorometric sandwich assay, wherein the antibody that recognizes p80 protein is a capturing antibody bound to a substrate, wherein the capturing antibody bound to the substrate is contacted with the sample, and wherein complex formation is measured by the binding of a tagged (colored molecules) secondary antibody that recognizes p80, wherein the antibody comprises a monospecific antibody that binds to a p80-specific amino acid epitope of p80 that ranges in length from 4 to 20 amino acids of the amino acid sequence described by SEQ ID NO:1.

In some embodiments, the antibody is a polyclonal antibody. In some embodiments, the antibody comprises a polyclonal monospecific/bivalent antibody for p80 and tubulin.

In some embodiments, the antibody comprises a monospecific antibody that binds to a C-terminal domain segment of p80 protein.

In some embodiments, the method of claim 1, wherein said disease comprises carcinomas, sarcomas, lymphomas and other malignant, pre-malignant, in situ, metastatic or other neoplasms, including leukemias, other blood cancers, fibromas, gliomas, or active proliferative diseases, or their precursor lesions.

In some embodiments, said tissue sample is selected from exfoliated cells, fragmented cells, tissue scrapings including pap smear, fluids/blood, urine, gut content, endoscopy, aspiration, biopsy, or endoscopic tool.

In some embodiments, said subject is human or animal. In some embodiments, said human is female or male, of any age, including the unborn.

In some embodiments, said method further comprising testing for the effects of surgical or medical treatment of said lesions.

Another aspect of the disclosure is directed to a method of using antibodies to identify the presence of lesions in cancer or proliferative disease, the method comprising: (a) obtaining a tissue sample from the subject; (b) diluting the tissue sample in a lysing buffer solution that makes the sample prepared for testing or prepares the sample for suitable morphologic study, such as wax impregnation and sectioning by a microtome, or other method of tissue preparation for histological and marker antibody study; (c) contacting the tissue sample with separate monovalent antibodies or a bivalent antibody that recognizes p80 and tubulin protein; (d) detecting complex formation or binding of the antibody to p80 and/or tubulin protein; and (e) wherein complex formation or binding is detected with an ELISA sandwich assay, wherein the antibody that recognizes the protein(s) is a capturing antibody bound to a substrate, wherein the capturing antibody bound to the substrate is contacted with the sample, and wherein complex formation is measured by the binding of a tagged secondary antibody that recognizes p80, wherein the antibody comprises a monospecific antibody that binds to a peptide epitope of p80/tubulin that ranges in length from 4 to 20 amino acids of the amino acid sequence of p80 described by SEQ ID NO:1.

In some embodiments, said method is performed in a clinical setting.

In some embodiments, said method detects the presence of p80 protein in the substantial absence of cross-reactivity with other proteins.

In some embodiments, a composition comprises said antibody.

Another aspect of the disclosure is directed to a method for diagnosis and treatment of a gynecologic disease comprising detecting an amount of p80 polypeptide consisting of SEQ ID NO: 1 in a human subject blood, serum or plasma sample from a human patient suspected of suffering from said gynecologic disease wherein the amount of p80 polypeptide is increased as compared to a control; and treating the subject to target the cancer.

In some embodiments, the amount of the p80 polypeptide is determined by contacting the human or animal sample with one or more antibodies or antigen binding fragments specific for full length human p80 under conditions suitable for polypeptide/antibody complexes to form and detecting the polypeptide/antibody complexes.

In some embodiments, detecting the amount of polypeptide is performed by an immunoassay selected from the group consisting of an enzyme linked immunosorbent assay (ELISA), western blot, immunofluorescence assay (IFA), radio immunoassay, hemagglutinin assay, fluorescence polarization immunoassay, microtiter plate assays, reversible flow chromatographic binding assay, and immunohistochemistry assay.

In some embodiments, the one or more antibodies or antigen binding fragments are detectably labeled.

In some embodiments, the one or more antibodies or antigen binding fragments are immobilized to a solid support.

In some embodiments, the one or more antibodies or antigen binding fragments are monoclonal antibodies, single chain antibodies, polyclonal antibodies, or antigen binding fragments (Fab fragments). These may be solely or as a cocktail.

Another aspect of the disclosure is directed to a method for detecting a p80 polypeptide defined by SEQ ID NO: 1 or any antigenic fragments thereof in a test sample, comprising: (a) obtaining a test sample from a patient; and (b) detecting whether p80 is present in the test sample by contacting the test sample with an anti-p80 antibody and detecting binding between p80 and the anti-p80 antibody.

In some embodiments, the step of detecting binding between p80 and the anti-p80 antibody comprises: (i) providing a reaction vessel, coated with a capture antibody onto its surface; (ii) adding a test sample comprising the target antigen into the reaction vessel to facilitate binding between the bound antibody and the target antigen; (iii) washing the solid substrate in the reaction vessel to remove any excess, target antigen not bound to the solid substrate; (iv) introducing the detection antibody into the reaction vessel to facilitate binding between the target molecules bound to the capture antibody and the detection antibody; (v) washing the solid substrate in the reaction vessel to remove any excess detection antibody not bound to the target molecule; and (vi) quantifying the amount of sandwiched target antigen by the presence of aggregated detection antibody-target antigen-capture antibody based on measurement of optical density.

By way of example and not limitation, cancer being detected or treated by the present disclosure may be atypical or malignant transforming, pre-invasive, non-invasive, invasive, and/or metastatic, hematologic, a solid tumor or solid tumor cancer of any organ or tissue, a carcinoma, a sarcoma, a myeloma, a leukemia, a lymphoma, or any combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. The consensus amino acid sequence for p80 based on “walking PCR”.

FIG. 2. Western blot of MAPIca in OVCA cell lines and OVCA clinical samples. Lane 1: SKOV3, lane 2: BIX3, lane 3: BIXLER, lane 4: DK2NMA, lane 5: ascitic OVCA cells, lane 6: papillary serous carcinoma, lane 7: ovarian epithelial carcinoma, lane 8: rat brain. An 80 kDa protein is present in lanes 2-8; the protein in lane 9 is 350 kDa.

FIG. 3. p80 expression in non-OVCA Cell Lines, p80 Expression in Breast & Endometrial Cancer Lanes: M: protein marker, 1: SKOV3 2: MCF7, 3: HEC.

FIG. 4. p80 in OVCA Specimens and Cell Lines Lanes: 1-3 OVCA tissue specimens, 4. Human brain, 5. DK2NMA-OVCA cells, 6. BIX3-OVCA cells, 7. SKOV3-OVCA cells, 8. BIXLER OVCA cells.

FIG. 5. Identification of p80 in OVCA lines by Western analysis after SDS-PAGE (4-15% gradient gel). Rat brain extract (lane a) used as a positive control indicated MAP 1 immunoreactivity corresponding to an MW value of 350 kDa. Immunoreactivity in the OVCA lines, SKOV3 (lane b), DK2NMA (lane c), BIXLER (lane d), and BIX3 (lane e), corresponded to a single band with an MW value of 80 kDa. MW=molecular weight markers (High Range SDS-PAGE standard).

FIGS. 6A-6C. (A) p80 was not expressed in 250 and 239 normal superficial cell lines. M=protein marker. Lane 1:239, lane 2:250, lane 3: SKOV3. (239 and 250 are cell lines of superficial obtained from normal ovary). (B) p80 was expressed in 249 OSEC cell line. M=protein marker. (1) 249, (2) 250 (3) SKOV3 (4) SKOV3 (5) 250 (6) 249 (7) SKOV3 (8) SKOV3, (9) 250, (10) 249. (C) Ovarian superficial 239 became positive after 44 days in culture. M=protein marker, (1): SKOV3 (2): 239. (3) 249, (4) SKOV3 (5) 239, (6) 249 (7) SKOV3, (8) 239, (9) 249. * To increase reliability, each sample was loaded in 6 wells. The picture shows triplicated results. ** SKOV3 is a positive control.

FIGS. 7A-7B. A mixing experiment to rule out the 80 kDa p80 being a degraded artifact. (A) Lane 1. 4 loaded only OVCA cell extracts. Lane 1: SKOV-3; 2: BIX; 3: Bixler; 4: DK2NMA. 5. Rat brain extract. 6-9. Rat brain+OVCA cells extracts. [Lane 6. Rat brain+SKOV 3. Lane 7. Rat brain+BIX3. Lane 8. Rat brain+BIXLER. Lane 9. Rat brain+DK2NMA.] (B) Surface plot analysis for Western blotting.

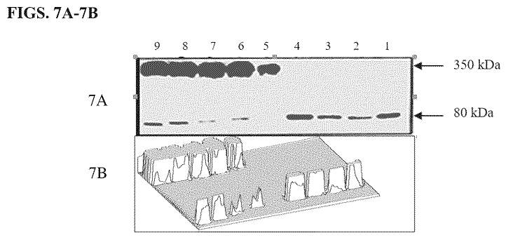

FIG. 8. Taxol binding assay for p80 and MAP1a in OVCA cells and rat and human brains. Microtubule-associated proteins co-precipitated with polymerized tubulin and were subjected to Western analysis. Lane 1: Protein standard. Lane 2 and 3: Taxol-treated pellet of rat brain. Lane 4 and 5: Taxol-treated human brain pellet. Lane 6 and 7: Taxol treated SKOV3 pellet. Lane 8 and 9: Taxol treated BIX3 pellet. 10. Supernatant from rat brain after three precipitations. 11 Supernatant from SKOV3 cells after three precipitations. 12: Human brain supernatant (non-treated) 13: SKOV3 supernatant (non-treated). Note: From lane 2-lane 5. The MW of the protein bands was >350 indicating the MAP1a protein. Lane 7-9. The MW of the protein c.a.80 kDa indicating the p80.

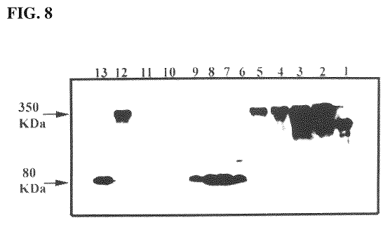

FIG. 9. Inhibition of expression of p80 by EM, TMX and DPI, as determined by Western analysis after SDS-PAGE (4-15% gradient gel). 24-hour treatments of SKOV3 (lanes a-c) with 10 μM EM (lanes a), 100 μM TMX (lanes b), and 10 μM DPI (lane c) produced significant decreases in the expression of p80, as compared to control DMSO-treated cells (lane d). Rat brain extract was used as a positive control (lane e). MW=Molecular weight markers (High Range SDS-PAGE standard). Actin was detected as loading control.

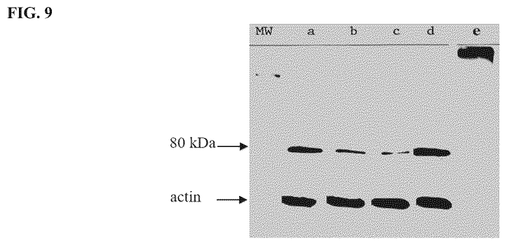

FIG. 10. SDS-PAGE/Western blot analysis of p80 in BIX3 OVCA cells following treatment with EGF, (see text). The lanes are as follows: (a) protein standards, (b) control-untreated, (c) EGF-treated. The density of p80 expression was increased after EGF treatment. Actin was detected as loading control.

FIG. 11. (Top): Light microscopy of THP-1 cells after treatment of PMA. A. untreated THP-1 cells; B. THP-1 cells treated with 10 ng for five days. (Middle): Western blotting analysis of p80 in PMA-treated THP-1 cells. M: protein markers, Lane 1. SKOV3 (OVCA) as p80 positive control, Lane 2. Untreated THP-1 cells, supernatant from the cell lysate. Lane 3. Untreated THP-1 cell, pellet from the cell lysate. Lane 4. 5 day-PMA (10 ng/ml) treated THP-1 cells, supernatant from the cell lysate. Lane 5:5 day-PMA (10ng/ml) treated THP-1 cells, pellet from the cell lysate. Lane 6: human brain protein extract as 350 kDa MAP1a control. Lanes 7-12 are duplications of Lane 1-7. Note: Before PMA treatment there is a thick 80 kDa band in Lane 2; after 5 days of PMA treatment, the 80 kDa band became undetectable. (Bottom): IF straining of THP-1 cells after 5 days treatment with PMA. A. Untreated THP-1 cells; B. PMA (10 ng/ml) treated THP-1 cells.

FIG. 12. Western blots of sera (S1-5) and ascitic fluid (as, A10) from advanced ovarian cancer patients using anti-MAP1a mAB appeared to show both free and tubulin-bound p80. Both 80 kDa and 130 kDa proteins were present in sera and ascitic fluid (red, blue, and green). A 300+kDa protein also is present in ascitic fluid (purple). p80 had a tubulin binding site but no actin binding site. No 122 kDa bands were seen. MAP1a has both tubulin and actin binding sites. No MAP1a was present in sera.

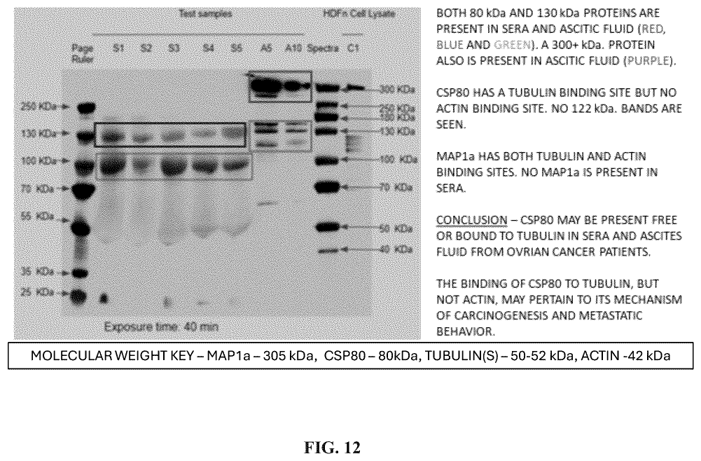

FIG. 13. Regular PAP staining diagnosis by morphology. Normal vs squamous cell carcinoma 40X. In normal cervical cells, the nuclei to cytoplasm ratio was similar from cell to cell. In cancer cells, the nuclei to cytoplasm ratio was processed from cell to cell.

FIG. 14. PAP smear re-stained for p80 (green fluorescence) and nuclei (blue dapi dye). Artifacts—much autofluorescence (the green background) is due to the adhesive used for slip cover on this actual pap smear slide after removal of the slide cover in order to stain the cells. Also, antibody was sequestered under the edges of cells, giving false impression of specific staining, the so-called “Edge Effect”.

### DETAILED DESCRIPTION OF THE DISCLOSURE

Currently, the oncogene/tumor suppressor gene mutation theory is a commonly accepted explanation of tumorigenesis. MAPs regulate cell growth and activities such as motility, invasion, size and rate of cell division. Just as these activities are what defines normal cells, abnormalities of MAPs could allow or cause abnormal cell activities or abnormalities of these activities. In the case of p80, there is a proven tubulin binding site present, but in contradistinction to normal MAP's there is no actin binding site; therefore, p80 might bind to tubulin and block the binding of normal MAP while triggering the formation of untethered (to actin) microtubules. Microtubules actively participate in forming the mitotic spindle that plays the key role in cell division, and abnormalities of the mitotic spindle may result in abnormal mitoses. Microtubules normally bind to the actin cytoskeleton that in turn is bound to the cell membrane via ezrin. The failure to connect the microtubules to the cell membrane could result in the anormal size and shapes of cells in cancer and proliferative diseases.

Using multidisciplinary platforms (differential transcriptomics, proteomics and bioinformatics), a new molecule was discovered in cancer cells but not in normal cells or tissues. p80 is a novel MAP, i.e., it binds to tubulin; however, p80 lacks the downstream actin binding site, which may explain its carcinogenic action. p80 is not present in normal cells/tissues, but is present in mesenchymal stem cells, in which it could play a role in embryonic or stem cell function.

The cells of cancer, and other proliferative diseases, develop abnormal mitoses and strikingly unusual shapes and processes, related to malfunctioning of their microtubule-cytoskeletal dynamics. Microtubule-associated proteins (MAPs) participate in these interactions. As a strategy to discover molecules specific to ovarian epithelial carcinoma (OVCA), MAP expression in OVCA cells and tissues was studied. The instant disclosure discloses the presence of a low molecular weight MAP that shares a high level of DNA sequence similarity and immunoreactivity with MAP1a but is unique to OVCA and other cancers but is not found in normal cells.

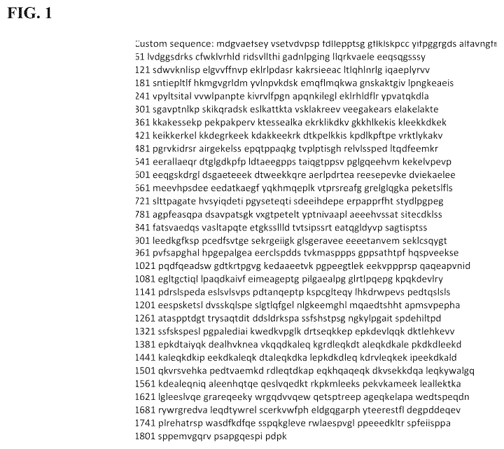

The present disclosure discloses the discovery of a MAP, termed p80, present in all cancers thus far studied, including OVCA and ENDOCA. p80 is absent in normal cells or organs. p80's amino acid composition has been derived by walking PCR plus computational DNA sequencing, FIG. 1. The findings regarding the presence of p80 and the absence of other MAPs are supported by immunohistochemical and Western analysis of cells, tissues, culture media and peritoneal fluid of OVCA, ENDOCA, and other cancer specimens.

In identifying p80, a Western Blot analysis was conducted on metastatic ovarian cancer specimens, normal human tissues and cancer specimens. All tissues were obtained under IRB-approved protocols. In this search for abnormal MAP-like proteins in cancers, normal human ovarian tissue, normal rat brain tissue and normal human brain tissues served as a MAP1a positive controls. A panel of anti-MAP monoclonal antibodies against the following proteins was used: MAP1a, MAP1b, MAP2a, MAP2b and anti-tau protein (Sigma, St. Louis), in the Western Blot analysis.

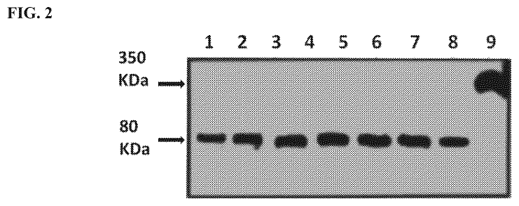

MAP1a was present as a single 350 kDa band in all tested brain extracts, FIG. 2. The anti-MAP1a immunostaining from OVCA extracts was restricted to a single 80 kDa band. No 350 kDa MAP1a immunoreactivity was found in the OVCA samples, despite its presence in concurrently analyzed normal brain tissue-positive control extracts (band 9, FIG. 2). None of the other anti-MAPs tested showed more than a single band. The 80 kDa protein was termed p80. p80 appears to be a unique protein that contains epitopes found in MAP1a (not shown).

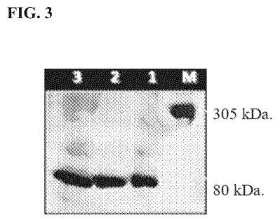

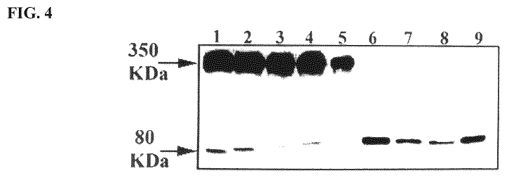

In multiple tests, no 80 kDa immunoreactivity was found in extracts of rat or human brain tissues or cultured rat or human glial cells, nor was it present in normal rat spleen, liver or lung (data not shown). On the contrary, p80 was the single band that was present in ovarian, breast and endometrial cancer, FIG. 3; FIG. 4.

The sequencing of p80 was conducted by “walking PCR primer extension”, also known as Directed Sequencing. This is a sequencing method for assessing DNA fragments that are too long to be sequenced using the chain termination method. Sequential overlapping primers were derived from consecutive authentic sequences from wild-type MAP1A and tested against p80 extracts. This method confirmed the presence of the complete MAP1a RNA sequence in normal brain tissues, but not in other, non-neuronal samples. On the contrary, overlapping primers revealed a smaller-sized transcriptome in extracts from OVCA. Starting with the MAP1a 5′ terminal the sequence obtained from OVCA (p80) was homologous with the WT until 2603 bp but stopped at that point. The amino acid sequence of p80 is provided in FIG. 1.

p80 was cloned using different sets of sense and antisense primers sequencing analysis was performed in both directions. To minimize ambiguities, overlapping analysis revealed major homology between the sequence of the human brain MAP1a and the p80 sequence thus far obtained from SKOV3 ovarian cancer cells (not shown). But the cDNA sequencing analysis shows that although there is similarity between 80kDa. p80 and the 350 kDa human brain MAP1a, insertion of four bp is present in the p80 cDNA that results in a premature stop codon and transcription of the mutated p80 protein. However, the origin of p80 is unknown at this time. It may be a result of a premature stop codon of MAP1a, or it may be a post-translational modification of the same. Regardless of the origin of p80, the discovery of its presence provides significant opportunity to detect, monitor and treat proliferative diseases and cancers.

FUNCTION OF p80 Additional efforts to determine the function of p80 led to further characterization, which revealed that the expression of p80 in cultured ovarian surface epithelial cells from normal-appearing ovaries was associated with transformation to malignant cells. p80 was not found in normal ovarian surface epithelial cells in tissue slices from normal ovary. This expression of p80 may be associated with the length of time in culture and the addition of estrogen and growth factor (insulin). Such an association would be consistent with the reported transformation of cultured ovarian surface epithelial cells from normal rat ovaries. These data were obtained in cultured cells; however, this may indicate an example of the above, that a gene is expressed that interferes with normal MAP's acting as tumor suppressing proteins. This would result in transformation or contribute to the transformation of normal cells and tissue to the pathology of a proliferative condition, tumor or cancer.

Samples of serum from women under treatment for advanced (Stage iv or v) ovarian cancer are shown in FIG. 2 to be marked on western blots challenged with an mAB against MAP1A, which contains 2603 amino acids highly homologous to p80. These findings substantiate that p80 and/or free-floating p80-containing structures present in bodily fluids such as ascitic fluid, endometrial secretion, blood or contents thereof, urine, serum, lymph fluid, endometrial washings from individual women, semen from men (p80 is expressed in prostate cancer) or washing from liquid biopsies or aspirates from the nipple of the breast or any of the body's cavities may reveal pre-malignant or malignant cells tissues in humans and animals, or a proliferative condition.

### Description of Agents to Discover and Monitor P80 and Diagnose a Proliferative Condition or Cancer

In one aspect, the disclosure provides a binding agent interacting with p80 in an individual with cancer or proliferative conditions. In another aspect of the disclosure, the binding agent interacting with, or binding to, p80 indicates the presence of a proliferative condition in a subject. In another aspect of the disclosure, the binding agent interacting with, or binding to, p80 detects the presence of a proliferative condition or a cancer or pre-malignant cells undergoing neoplastic change and is used for diagnosis or monitoring of a proliferative condition or cancer.

Various p80 protein binding agents may be used in the instant disclosure. For example, the binding agent may be an antibody, or a functional fragment thereof. Antibodies (monoclonal antibodies, mAB) are amino acid-literate molecules that find and bind to “epitopes”, small groups of amino acids, that uniquely identify individual proteins. Monoclonal antibodies are specific in their interaction with a single, protein unique epitope. “Functional fragment” includes a fragment that binds the antigen, preferably binds the antigen and has at least one functional effect of the full antibody (such as inhibiting the function of the antigen or binding partner), especially when used in the context of the subject treatment method. However, functional fragments may only need to be able to bind their intended target molecule for the various diagnosis embodiments of the disclosure. The antibody may be a polyclonal antibody or a monoclonal antibody. The antibody may be a xenogeneic, an allogeneic, or a syngeneic antibody. The antibody can also be a modified antibody selected from the group consisting of a chimeric antibody, a humanized antibody, and a fully human antibody. The functional fragment of an antibody may be F(ab′)2, Fab, Fv, or scFv, one or more CDRs, etc. Such “humanized anti-bodies” also may be anti-cancer agents, as they may be administered to cancer patients and will seek, bind to p80, thus inactivating it.

Generating monoclonal antibodies may be via in vitro or in vivo methods. Whereas protocols for generating monoclonal antibodies are well known in the art, a general description is provided by way of example and not limitation. Those skilled in the art may leverage varying protocols or organisms to the same end.

Generally, in vivo methods entail immunizing an animal, such as a mouse, rabbit or other animal, with a suitable antigen, p80, or an epitope thereof that may be desired for detection. The antigen is injected with an adjuvant, such as Freund's adjuvant. This leads to production of desired antibodies in the animal's body. The antigen may be injected into the animal multiple times. Typically, this immunization is done for a few weeks until the antibody concentration in the animal increases to the desired level. After a desired number of weeks, the blood, or contents thereof, or spleen, is obtained from the animal to assess the antibody titer, using techniques known in the art, such as ELISA or flow cytometry.

Monoclonal antibodies-A general description of in vitro methods is provided by way of example and not limitation. Generating monoclonal antibodies involves fusing activated antibody-producing B-cells with myeloma cells, to form “a hybridoma.” These hybridomas have immortal grown properties of the myeloma cell and can secrete antibodies due to their B-cells. As described above, an animal previously immunized against a p80, or an epitope thereof, has an enhanced population of B-lymphocytes that produce antibodies against the antigen. The hybridoma cell line is cultured and is screened for the hybrids producing the desired antibodies. Screening may be performed by standard methods in the art, such as ELISA. The hybridoma cells may be cultured in vitro, or may be injected into the peritoneal cavity, in which case the ascitic fluid will contain high concentrations of monoclonal antibodies against p80.

Monoclonal antibodies compatible to humans (humanized) can target human proteins such as p80 in vivo without adverse effects on patients. The same process is performed for the safe treatment of animals.

Monospecific antibodies are antibodies that target a specific antigen. They are either monoclonal antibodies or polyclonal antibodies that have been purified by affinity chromatography using the antigen as ligand. This means that a polyclonal monospecific antibody raised against a synthetic peptide can be a timesaving, cost effective and highly specific alternative to a monoclonal antibody. Monospecific antibodies compatible to humans (humanized) can target human proteins such as p80 in vivo without adverse effects on patients. The same process is performed for the safe treatment of animals.

Antibodies to p80 may be used to measure these proteins in biological fluids and tissues using immunoassay that depends on the specificity of the monoclonal antibody. In the case of p80, the expression in cancers is unique and proves the involved cells or their fragments to be malignant.

Antibodies to p80 may be used to mark the proteins in specimens from test tissues. Conventional immunohistochemistry, known in the art, is carried out for this purpose. Mass spectroscopy and Western blotting are also used for detection in sample tissues.

A Point-of Service test. ELISA (enzyme-linked immunosorbent assay) testing may be used for rapid qualitative diagnosis, in the format of conventional pregnancy or covid immune tests. Providing an absorbent solid substrate, having a location to receive a biological sample potentially comprising p80 or a fragment thereof, the sample spreads on the absorbent solid substrate to interact and potentially bind to a mobile antibody, the conjugate further mobilizing to an immobile antibody, to form a p80 antigen sandwich, the immobile antibody providing a color change on the absorbent solid substrate in the presence of p80 or a fragment thereof. The monoclonal antibodies used in this assay may bind to p80 or any p80 epitope-containing fragment thereof.

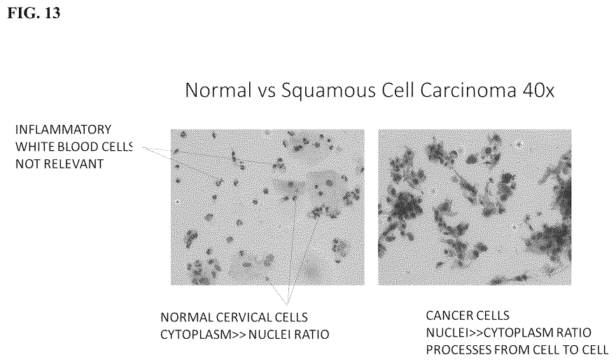

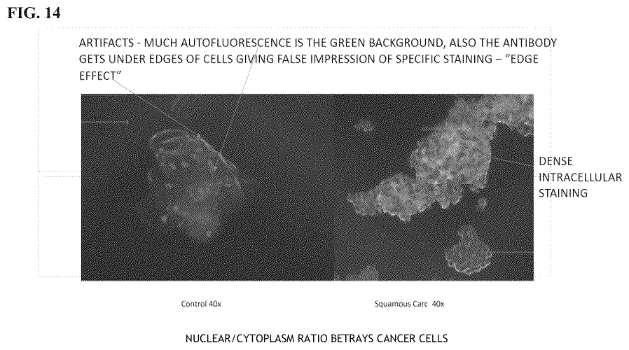

Non-Point-of-Service testing for p80. In certain instances, it may be necessary for the tests to be carried out elsewhere than the office. Generally, these tests will be employed because of need for special equipment, such as microscopes and chromatographic techniques, or special skills, such as performance and interpretation of immunostaining of desquamated cells, such as pap smears, bronchial and endometrial washings (FIG. 13 and FIG. 14). In the case of liquid biopsies such as blood or other bodily fluids immunoassays using monoclonal antibodies to p80, mass spectrometry, western blotting, etc. can be employed. The same applies to immunostaining of tissues or desquamated cells.

The instant disclosure provides a method to use the binding agents, such as monoclonal antibodies or small molecules, as detection agents for detecting and/or quantitating the p80 proteins in a number of pathological conditions, using samples such as body fluids, including but not limited to, peritoneal fluid, ascitic fluid, endometrial secretion, blood, serum, urine, semen, lymph fluid, aspirated fluid or scraped, lysed cells from the vagina/cervix, endometrial cavity or any other body cavity, including the gastrointestinal, urologic, breast duct, oropharynx and the like, which are obtained from an individual suffering from cancer, malignant or pre-malignant change such proliferative conditions, or at risk of developing such conditions. This is termed cancer screening. Using specific antibodies, the sensitivity and specificity of the subject method are high, while the false positive rate is low. The salient issue for the instant disclosure is that these conditions are marked by the presence of p80 while normal tissues are not. This use for finding abnormal tissues does not exclude the possibility that other conditions than mentioned will express p80 and therefore be identified. The instant disclosure is broadly applicable to conditions identifiable by the presence of p80 and is not only limited to the conditions listed by way of example in the instant application. As a result, the subject method can detect a low level of true positive signal, thus providing a method for early detection/screening and diagnosis of diseases where early screening/diagnosis is critical for prognosis.

The diagnostic method of the instant disclosure not only provides an early diagnosis/screening means for certain proliferative diseases, but also provide a non-invasive means to monitor the progress of the disease over time, its responsiveness to various treatments, and/or the possible recurrence of diseases previously in remission. Thus, the term “diagnosis” includes not only the initial diagnosis but also the monitoring of disease progression, the response of the disease to specific treatment regimens, the detection of possible recurrence, and screening of healthy individuals or individuals at high risk of developing the subject disease conditions, etc.

In the instant disclosure, p80 monoclonal antibodies are bound (affixed) to fluorescent or imaging-opaque molecules for the purpose of imaging to find their presence at the surgery or using imaging. This is for the purpose of clinical identification, clinical staging for treatment or to evaluate the results of treatment.

As described below, humanized antibodies may also be bound to anti-cancer and proliferative drugs for the treatment of cancer or proliferative diseases. Monoclonal antibodies can be bound to nanoparticles and nanospheres by the monoclonal antibodies targeting the p80 in cancer or another proliferative disease. The nanoparticles may contain pharmaceutical treatments, radioactivity or other modalities for the treatment as well as the mapping of these conditions.

A novel two antibody point-of-service test for p80. In a second, supportive embodiment, a Point of Service test of the authenticity and functionality of p80 may be utilized; a combined anti-p80 and anti-tubulin assay. p80 contains a tubulin binding site. It is shown that this binds tubulin in vitro and that tubulin is present in a portion of the p80 identified in body fluids. Since the p80-specific epitope (consecutive amino acids specific for p80) is not obscured by the tubulin binding site nor does bound tubulin interfere with the p80 binding, it is possible to furnish a novel, two-protein assay for the presence of p80 in which the solid membrane upon which the bodily fluid, etc. can be absorbed and through the impregnation with both the mAb for p80 and the mAb. Not only would this reveal the presence of p80, while the biological implications are to be tested, they are of interest regarding activity of neoplastic activity of p80, possible points for anti-p80 treatment and will strengthen imaging techniques for clinical staging and location of sub-clinical metastases.

In some embodiments, the Point of Service test is a method that includes a prepared platform such as an absorptive tape, or a similar wet or dry absorbent surface that conducts the digest of the sample which can be put on one end of the tape and as it passes through first a vehicle control that shows the line that any control sample would, and then through the prepared marker p80 binders and enzymatically triggered marker dyes. If the platform is liquid, the marker will be a color change driven by a system of antibodies analogous to the dry method, constituting a test that can be executed at the point of service (Point-of Service test; POS). POS can be loaded with specific amounts of signaling agents to allow quantification of the p80 present in the sample. An alternative similar platform test is constructed to have both anti-p80 and anti-tubulin to detect and quantify the presence of p80-bound tubulin.

### Small Molecules

The binding agent can be a small molecule antagonist of the p80 protein, such as those with molecular weights no more than about 5000 Da, 4000 Da, 3000 Da, 2000 Da, 1000 Da, 500 Da, 200 Da, or less than 100 Da. Such small molecule binding agents may be small peptides, or peptide-mimetics, or any other organic or inorganic compounds that can bind any p80 epitope, with or without the presence of bound tubulin, and inhibit the protein function (such as p80′s role in proliferation and/or invasion, metastasis).

Computational modeling or other means may reveal extant and synthetic molecules (small molecules) that interact with p80 amino acid structures, with or without bound tubulin. The small molecules may bind to or interfere with the conformation or function of p80 or be attached to nanoparticles with cargos as described herein. In the case of direct action these small molecules are considered pharmaceutical agents in the instant application.

Monoclonal antibodies and small molecules against p80 may be attached to anti-cancer agents or nanoparticles to approximate them to pre-malignant, malignant, or proliferative disease cells. In such cases the therapeutic effects may be direct. Alternatively, the monoclonal antibody, small molecules or nanoparticles may be internalized, e.g., by endocytosis, after which they can target and attack abnormal protein expression or the cells that express p80.

### EXAMPLES

Various aspects of the instant disclosure are described below. The following examples are for illustrative purposes only and should in no way be construed as limiting in any respect of the claimed disclosure.

### Example 1

Serum Sample Preparation for testing: Each sample was concentrated to 2 mL or less by centrifuging each sample in a five kDa concentrator at 14,000 g for over two hours. Albumin was depleted using the Albumin Depletion kit (Pierce, catalog no. 85160) according to the manufacturer's protocol. The protein concentration was quantified by Qubit® fluorometry method (ThermoFisher, Rockville, MD). 20¬μg of each was processed by SDS-PAGE using a 10% Bis-Tris NuPAGE mini-gel (Invitrogen) with the MES buffer system, the gel was run approximately 2 cm. The mobility region was excised into 20 equally sized bands and processed by in-gel digestion with trypsin using a ProGest robot Digilab and washed with 25 mM ammonium bicarbonate followed by acetonitrile. Then reduced with 10 mM dithiothreitol at 60° C. followed by alkylation with 50 mM iodoacetamide at RT; and digested with trypsin (Promega) at 37° C. for 4 h, followed by quenching with formic acid. The supernatant was analyzed directly without further processing.

Mass Spectrometry: Half of each digested sample was analyzed by nano LC-MS/MS with a Waters M-Class HPLC system interfaced to a ThermoFisher Fusion Lumos mass spectrometer (ThermoFischer, Rockville, MD). Peptides were loaded on a trapping column and eluted over a 75 μm analytical column at 350 nL/min; both columns were packed with Luna C18 resin (Phenomenex). The mass spectrometer was operated in data-dependent mode, with the Orbitrap operating at 60,000 FWHM and 15,000 FWHM for MS and MS/MS respectively. The instrument was run with a 3 s cycle for MS and MS/MS. 10 hrs of instrument time has used the analysis of each sample.

Data Processing: Data were searched using a local copy of Mascot (Matrix Science) with the following parameters:
Enzyme: Trypsin/P;
Database: SwissProt Human (concatenated forward and reverse plus common contaminants);

1Fixed modification: Carbamidomethyl (C);
Variable modifications: Oxidation (M), Acetyl (N-term), Pyro-Glu (N term Q), Deamidation (N/Q);
Mass values: Monoisotopic;
Peptide Mass Tolerance: 10 ppm;
Fragment Mass Tolerance: 0.02 Da; and
Max Missed Cleavages: 2.

Mascot DAT files were parsed into Scaffold (Proteome Software) for validation, filtering and to create a non-redundant list per sample. Data were filtered using 1% protein and peptide FDR and requiring at least two unique peptides per protein.

### Example 2

### Serum Samples (MS1302)

Sample Preparation: Samples were pooled per client's instructions. Each serum sample was depleted using Proteome Purify 12 Human Serum Protein Immuno-Depletion Resin (R&D Systems, Catalog no. IDR012-020) according to the manufacturer's protocol. Depleted samples were buffer exchanged into water on a Corning Spin X 5 kD molecular weight cut off spin column and quantified by Qubit fluorometry (Life Technologies). 50 μg of each sample was reduced with dithiothreitol, alkylated with iodoacetamide and digested overnight with trypsin (Promega), and washed with 25 mM ammonium bicarbonate followed by acetonitrile. Then samples were reduced with 10 mM dithiothreitol at 60° C. followed by alkylation with 50 mM iodoacetamide at RT and digested with trypsin (Promega) at 37° C. for 4 h. The samples were quenched with formic acid and the supernatant was analyzed directly without further processing.

Mass Spectrometry: 2 μg of each sample was analyzed by nano LC-MS/MS with a Waters M-Class HPLC system interfaced to a ThermoFisher Fusion Lumos mass spectrometer. Peptides were loaded on a trapping column and eluted over a 75 μm analytical column at 350 nL/min; both columns were packed with Luna C18 resin (Phenomenex). A 4 hr gradient was employed. The mass spectrometer was operated in data-dependent mode, with the Orbitrap operating at 60,000 FWHM and 15,000 FWHM for MS and MS/MS respectively. The instrument was run with a 3 s cycle for MS and MS/MS.

Data Processing: Data were searched using a local copy of Mascot (Matrix Science) with the following parameters:
Enzyme: Trypsin/P
Database: SwissProt Human (concatenated forward and reverse plus common contaminants and appended with a custom sequence, see below)
Fixed modification: Carbamidomethyl (C)
Variable modifications: Oxidation (M), Acetyl (N-term), Pyro-Glu (N-term Q), Deamidation (N/Q)
Mass values: Monoisotopic
Peptide Mass Tolerance: 10 ppm
Fragment Mass Tolerance: 0.02 Da
Max Missed Cleavages: 2

### Example 3

### Enzyme Linked Immunosorbent Assay (ELISA)

The assay may be done in various ways, including an Enzyme Linked Immunosorbent Assay (ELISA), in which a first immobilized binding agent (e.g., immobilized on a solid surface such as a 96-well plate, etc.) was used to bind and isolate p80 in a fluid sample, and a second detection binding agent (such as a binding agent labeled by a fluorescent dye, an enzyme, or a radio label) was used to bind the bound p80 protein. The presence and amount of the labeled second detection binding agent may then be determined/measured.

According to the subject method, the amount and/or concentration of the p80, or fragment thereof, detected in the sample was proportionally indicative of the severity and/or extent of the proliferative condition.

The diagnosis method of the disclosure may be performed, e.g., the amount and/or concentration of p80 was determined, using a binding agent which binds the p80. The binding agent may be an antibody, or a functional fragment thereof. “Functional” may only require the ability to bind in the context of the subject diagnosis methods. The antibody may be a polyclonal antibody or a monoclonal antibody. The antibody may be a xenogeneic antibody, an allogeneic antibody, or a syngeneic antibody. The antibody may be a modified antibody selected from the group consisting of a chimeric antibody, a humanized antibody, and a fully human antibody. The functional fragment may be F (ab, Ä≤)2, Fab, Fv, scFv, or one or more CDR's.

In certain embodiments, the binding agent may also be tagged by a label, such as a fluorescent label, an enzyme label, or a radiolabel.

The diagnosis methods of the disclosure may be used to detect p80 and fragments thereof.

In certain embodiments, the p80 binding agent may be labeled by a moiety, such as a fluorescent dye, an enzyme, or a radio-imaging reagent.

Sandwich Assay for ERM protein Detection/Quantitation.

A sandwich ELISA assay was used to detect and/or quantitate p80 in tissue sample/fluids. For example, to detect/quantitate p80 in a sample, binding agents such as a p80 capture antibody were bound to a 96-well plastic plate or absorbent solid substrate (or other solid support). p80 in samples was then captured and then detected/quantitated by a specific antibody. The third element in the “sandwich” was a species-specific anti-IgG that is labeled with an enzyme, such as peroxidase. The peroxidase reaction was developed and quantitated by an ELISA plate reader.

### Example 4

Cell cultures: A. OVCA cell lines: Five OVCA cell lines (SKOV3, BIX3, BIXLER, DK2NMA and CAOV3) were used. The cell lines were being maintained in a complete culture medium composed of DMEM supplemented with 10% fetal calf serum and penicillin, streptomycin and fungizone. The culture medium was refreshed twice per week. Sub-confluent cultures are used for experiments. B. Normal ovarian epithelial cells: A) Normal human ovarian epithelial cell lines taken from pre-/postmenopausal human ovaries (courtesy of Dr. B. Karlan) (35) were grown in 199/MCDB 105 Media supplemented with penicillin and streptomycin without anti-fungal antibiotics. Cell culture media were refreshed twice per week. Sub-confluent cultures are used for experiments. B) Cultured surface epithelial cells and (transformed) metaplastic, i.e., cuboidal cells from ovarian clefts with microvilli on their luminal borders.

Clinical specimens: A) Fresh epithelial ovarian cancer (OVCA) specimens were obtained, on ice, from unrelated gynecological operative procedures. Confirmation of the diagnosis and characterization of the important histological characteristics were furnished by the pathology department. B) Samples from primary and metastatic samples were furnished on ice. In all cases the clinical history was available.

Immunofluorescence Microscopic Studies: This method was adapted for the detection and observation of tubulin-and MAP antigens within cells. Cells were plated on polylysine precoated chambers (Falcon, Becklon, Dickinson) at a density of 2×10⁴ cells/100 μl. Cells were incubated in a CO₂ incubator at 37° C. for 24 hours to allow attachment. Cells were washed with microtubule stabilization buffer (MSB: 0.1M PIPES, 0.5 mM MgCl₂, 5 mM EGTA, 0.1% aprotinin, 0.1% PMSF, 0.1% leupeptin, 0.1% soybean trypsin inhibitor, pH 7.2) for 1 min at 37° C. and immersed in MSB, containing 1% paraformaldehyde, 0.25% glutaraldehyde and 0.5% Triton X100, for 4 min at 37° C. Proteins were labeled by indirect immunofluorescence using monoclonal antibodies to MAP1a (p80) or tubulin. Second (indicator) antibodies were rhodamine-conjugated anti-mouse IgG and fluorescein-conjugated anti-mouse IgG. Double labeling studies were carried out using a mouse mAb to MAP1a and rabbit polyclonal Ab to tubulin, second antibodies were anti-mouse IgG conjugated FITC and anti-rabbit IgG-conjugated rhodamine. The method control absented the primary antibodies. Cells were evaluated using confocal microscopy and the data processed using the statistical packages on the microscope.

Western Blot Analysis: The cell monolayer was lysed with lysis buffer containing six protease inhibitors (Protease Cocktail; Roche), incubated on ice for 10 min, and scraped with a rubber policeman. Cell lysate was collected and centrifuged at 12,000× g for 30 min. Supernatants were saved for SDS-PAGE and Western blot analysis, and pellets were extracted by SDS-PAGE sample buffer (minus reducing reagent). The total amount of protein in each extract was determined by the bicinchoninic acid (BCA) method. SDS-PAGE was performed, loading the same amount of total protein in the same volume into each sample well, using commercially available precast 4-15% gradient SDS-polyacrylamide gels (Bio Rad). High range SDS-PAGE standard (Bio Rad) was run as a standard. After electrophoresis, proteins were transferred onto a PVDF membrane. After transfer, the membrane was incubated with primary antibody, then washed, and incubated with peroxidase-labeled second antibody. Signals were visualized on Hyperfilm-ECL by the ECL-reagents (Amersham).

RT-PCR: This technique is the most sensitive and specific for the evaluation of gene expression. In principle, it can detect as little as one cell's RNA. It was used to determine minor expression of p80 or MAPS, in case only a small sample was available, such as a few cells obtained from needle aspiration. Method: Total RNA was extracted with Trizol reagent (Life Science Technologies). The reverse transcription reaction (RT-reaction) was carried out using the cDNA synthetic kit (Pharmacia). PCR reaction was carried out by using a kit purchased from Roche and ìhot started by anti-Tag polymerase monoclonal antibody (Sigma). Thermocycler reactions was performed with a 2400 PCR thermocycler (Perkin-Elmer). PCR product was identified using 1% agarose gel electrophoresis followed by ethidium bromide straining, with signals visualized with a UV-illuminator. In some cases when DNA sequencing was needed, pfu DNA polymerase (Strategen) was used instead of the other DNA polymerases because of higher fidelity. The DNA cloning allowed the design of highly specific primers and internal competitors for quantitative determination of p80.

Northern Blotting Analysis: Total RNA was extracted by using Trizol reagents (Gibco Life Technologies). RNA electrophoresis was performed on a 1% agarose formaldehyde denaturation gel. RNA was transferred onto a nylon-membrane by capillary transfer. The ECL direct nucleic acid labeling and detection system (Amersham) was used for the prehybridization/hybridization, and the signal development of RNA. In this system, the DNA probe was labeled with covalently conjugated peroxidase. Signals were developed by ECL reagents. The intensity was measured by densitometry. This assay is more specific than Western blotting when specific primers are used. Under stringent control, this technique can also be used for quantitative determination of p80 gene expression.

SDS-PAGE and Western Blot Analysis: The cell monolayer was lysed with lysis buffer containing 6 protease inhibitors (Boehringer-Mannheim). Cell lysate was collected by centrifugation after the supernatants were saved. The pellet was extracted by boiling with SDS-containing buffer. The total amount of protein in each extract was determined. SDS-PAGE was performed by loading the same amount of total protein into each sample well. After electrophoresis, proteins were electro-transferred onto a PVDF membrane. Then, the membrane was incubated with primary antibody, washed, and incubated with peroxidase conjugated horse anti-mouse IgG (H+L). Signals were developed with an enhanced luminescence reagent (ECL, Amersham) and visualized on Hyper ECL-Film. (Amersham). Signal density was measured and analyzed by a densitometer. Western analysis can furnish information on immunoreactivity and molecular size; therefore, this technique was often used for the first-line screening of the presence of p80. However, if there was any ambiguity about the cross-reactivity, the results were double checked with Northern blotting or RT-PCR with specific primers.

### Example 5

Culture of OVCA cell lines and cell cultures. Rationale: In order to determine the efficacy of inhibiting human OVCA cells with drugs, four extensively characterized OVCA cell lines, three in-house lines, DK2NMA, BIXLER, BIX3 (all courtesy of Drs. S. Chambers and B. Kacinski), (Peter B. Kaufman, William, Wu, Donghern Kim, Leland J. Cseke. Molecular Biology and Medicine. CRC Press inc., Boca Raton, Florida Chapter 12:289-304, 1995) and SKOV3, obtained from American Type Culture Collection, were established in mono-layer culture. Experiments: In preparation for drug treatment, cell suspensions were thor-oughly dispersed, precisely counted and then seeded into another flask at the desired cell den-sity. Conclusion: The cell lines were established, and test drugs shown to predictably affect the cells. Results were produced that meet stringent reliability criteria, including precision i.e., small variability between replicates.

### Example 6

Inhibition of OVCA cell proliferation by EM (estrogen mustard). Rationale: Clini-cally, EM has modest systemic toxicity and has been partially effective in the treatment of advanced prostatic carcinoma. Previously glioblastoma cells showed that EM/E-CC arrested the cancer cells in G2/M and killed cancer cells in culture. Therefore, the effect of EM on OVCA cells was studied. Experiments: 3H-thymidine uptake was used to test the effect of EM against cultured OVCA cells in a wide range of concentrations (1-100 μg/ml). Findings: EM caused a marked, dose-dependent inhibition of 3H-thymidine incorporation for all OVCA cell lines. Conclusions: (1) EM blocked proliferation and suppressed DNA synthesis in vitro in OVCA, justifying further studies using it as an agent to target MAP's in OVCA. (2) These studies did not assess initially sublethal effects of EM; therefore, the Colony Form-ing Assay was performed as described below (Attacking cancer. USA Today Article. May 19, 1998). As well, to establish the mechanism of cell death several different tests for apoptosis were performed (Perez R P, Godwin A K, Hamilton T C. Ozols R F. Ovarian cancer biology. Seminars in Oncology. 18 (3): 186-204, 1991).

### Example 7

Effect of EM on the cell cycle of OVCA. Rationale: Since EM's antiproliferative activity could be due to the disruption of mitosis and other crucial microtubule-dependent cellular functions, flow cytometry was used to investigate the effect of EM on the cell cycle distribution of OVCA. Experiments: Following EM administration (1-100/ μgm/ml) the flasks were trypsinized and all cells subjected to flow cytometry in the Yale Cancer Center Core laboratory. In all OVCA cell lines, among the living cells, EM treatment for 24 hours resulted in a significant increase in the percentages of cells in G2/M phase as compared to control, DMSO-treated cells. Conclusions: (1) EM induced cell cycle synchronization at G2/M. This was consistent with an action on OVCA microtubule associated proteins (MAP's). (2) Synchronization in G2/M raised the possibility of radio-sensitization (Barnes WM. PCR amplification of up to 35-Kb DNA with high fidelity and high yield from lambda bacteriophage templates. Proc Natl Acad Sci USA March 15; 91 (6): 2216-20, 1994; Mandelkow E, Mandelkow E-M. Microtubules and microtubule-associated proteins. Current Opinion in Cell Biology 7:72-81, 1995.

### Example 8

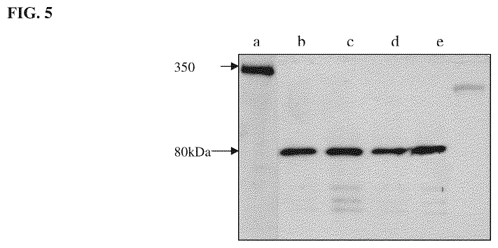

Identification and characterization of p80 in OVCA by Western Blotting. Rationale: It has been reported that EM binds to certain MAP's, (Desai A, Mitchison T J. Microtubule polymerization dynamics. Ann Rev Cell Dev Biol. 13:83-117, 1997; Riederer B, Cohen R, Matus A. MAP5: a novel brain microtubule-associated protein under strong developmental regulation. J. Neurocytol. 15:763-775, 1986) preventing microtubule assembly and thereby inhibiting cell division. Therefore, OVCA cells were studied to determine their MAP-content. Experiments and Findings: Total protein from all four OVCA cell lines was extracted under strictly controlled conditions: All the extraction procedures were performed on ice and the extraction buffer was supplemented by six protease inhibitors (Protease Inhibitor Cocktail, Boehringer-Manheim). Proteins were separated by SDS-PAGE. On Western analysis, a single band was demonstrated in each OVCA line, corresponding to a MW of approximately 80 kDa., which reacted strongly with anti-MAP1a mAb (FIG. 5). In comparison, in the control, identically extracted rat brain, the single band found reacted with the MAP1a mAb, but it corresponded to a MW of 350 kDa. Thus, the rat brain MAP1a control was consistent with the MAP1a described previously, but there was a MAP1a-like protein in OVCA that had a MW only one fourth that of MAP1a. Conclusions: (1) The 80 kDa. p80 appeared to be a native protein that may be specific for OVCA. In fact, treatment of glioblastoma cells with EM appeared to affect MAP 5, while EM affected the p80 in OVCA. Thus, these studies were compatible with the notion that there might be specifically MAP-enriched tissues which responded characteristically to EM/E-CC. But they did not rule out effects of EM on other, perhaps yet undiscovered MAPs. (2) The moderate side effects in EM-treated subjects with prostate-and other cancers implied that the MAP's in normal cells were only modestly affected by EM.

### Example 9

Absence of p80 on Western blotting of normal tissues. Rationale: To determine whether p80 was a cancer-specific MAP Western blotting of extracts of representative normal human tissues gathered at surgery were used and verified by pathologic evaluation. Experimental: Tissues were prepared by our standard Western blotting technique and tested with the same antibody that binds the 350 kDa. MAP1a in brain. Further, SKOV3 cells furnished a positive control for the 80 kDa. p80. Results: 10 μg of protein from normal human amygdala, placental, ovary, endometrium, uterine leiomyoma, and cultured astroglia were studied. Although the brain showed 350 kDa. MAP1a only (negative control) and the SKOV3 cells contained only p80 (positive control), none of the other normal tissues revealed p80. Because the results were negative, no illustration was shown. Comment: The normal tissues tested did not express p80.

### Example 10

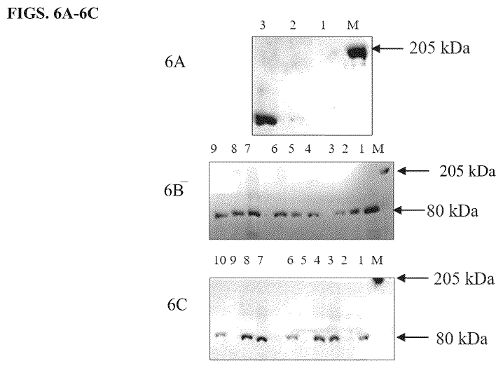

Absence of p80 in primary cultures of human superficial ovarian epithelial cells and appearance of p80 in latter passaged ovarian epithelial cells. Experimental: The expression of p80 and MAP1a were examined using Western blotting in 3 epithelial cell lines (239, 249, 250) from normal ovaries (gifts of Dr. B. Karlan). Results: It was found that 249 was p80-positive from the first culturing in the laboratory, but on microscopy 249 had multinuclear cells and rapid proliferation, similar to SKOV3 cells. Cell lines 239 and 250 began with normal phenotypes and were p80-negative. They had slow growth rates. None of these cell lines became positive for MAP1a. After an additional 44 days of culture, cell line 239 became positive for p80 while 250 remained negative. Both 239 and 250 continued to grow slowly and apparently demonstrate normal phenotypes. Conclusion: These three superficial cell lines were obtained from “normal” ovaries. In light of previous reports of “spontaneous” malignant transformation of “normal” rodent OSEC in culture, it is important to evaluate each cell line's history and length of culture prior to transfer. The present data was consistent with the hypothesis that p80 was associated with malignant transformation. However, molecular probes are needed to better assess the expression of MAP's in these cell lines (FIG. 6).

### Example 11

A mixing experiment to rule out the p80 being a degradation artifact. Rationale and Experiments: To determine whether p80 was a degradation artifact of high molecular weight MAP1a or a novel MAP1a-like protein, a mixing experiment was carried out. The cell lysate was mixed from brain tissue, which contained the well-characterized MAP1a (350 kDa), with the cell lysate from OVCA, which contained the lower molecular weight p80. Western analysis of the mixed-sample lysate with that of the individual lysates were compared. Findings: In the mixed-sample lane, the MAP1a epitope was still found in two easily separable bands; one was 350 kDa and the other was 80 kDa; i.e., the same findings as with single-sample lysates. The intensity of the 80 kDa. was not increased, indicating that in the experimental conditions, the 350 kDa. MAP1a was not degraded to 80 kDa. p80 (Joshi HC. Microtubule dynamics in living cells. Current Opinion in Cell Biology 10:35-44, 1998). Conclusions: p80 was a novel protein, it was not a degraded artifact of the 350 kDa. MAP1a protein.

### Example 12

Taxol-binding assay for p80 in OVCA cells. Rationale and Experiments: Using Western analyses, it was shown that there was a 80 kDa protein in OVCA cells with MAP1a immunoreactivity (p80). To determine whether this protein was truly a microtubule-associated protein, a Taxol-binding assay was conducted (Karlan B Y, Baldwin R L, Lopez-Luevanos E, Raffel L J, Barbuto D, Narod S, Platt L. Peritoneal serous papillary carcinoma, a phenotypic variant of familial ovarian cancer: Implications for ovarian cancer screening. Am J Obstet Gynecol 180:917-28, 1999): Rat or human brain and OVCA cells were homogenized in microtubule assembly buffer (0.25 M sucrose, 100 μM MOP, 0.5 μM MgC12, 1 mM EGTA, 1 mM GTP plus protease inhibitor). Because the homogenates contained some microtubules that had already combined with MAP's, the homogenates were centrifuged, and the supernatant used for the binding assay. The supernatant containing soluble tubulin and unbound MAPs was incubated with Taxol to polymerize the tubulin into microtubules, which co-precipitated with MAP's. After extensive washing, the pellets were resuspended in SDS-PAGE sample buffer and subjected to SDS-PAGE and Western analysis. The Western blot membrane was probed with mab-MAP1a and developed with ECL-reagents. Findings: In OVCA samples only a □80 KD MAP (p80) co-precipitated with tubulin, while in human and rat brain samples a 350 KD MAP1a was co-precipitated. Conclusion: The p80 was a true and specific microtubule-associated protein, being bound to microtubules in OVCA, but not in brain.

### Example 13

Partial sequencing of p80: The published human MAP1a genomic and mRNA sequence (Schwende H, Fitzke E, Ambs P, Dieter P. Differences in the state of differentiation of THP-1 cells induced by phorbol ester and 1,25-dihydroxyvitamin D3. Journal of Leukocyte Biology, 59:555-61, 1996) was used to design primers and performed RT-PCR and sequencing analysis. Experimental: RNA was extracted by Trizol reagent (Life Science Technologies). Purified total RNA was digested by RNase-free DNase to eliminate genomic DNA contamination. First strand cDNA was synthesized by a Ready-to-go cDNA synthesis Kit (Amersham-Pharmacia). RT-PCR was performed by using the following primer sets: 1) ZCMP1 (sense: ggg aga cct cat cct aca ga; anti-sense: tgc tcc tcc tct tag ctc gc, ZCMP2: sense: atc tgg act tcc gtt ac, anti-sense: tag cac ggc tcc tct cta tt and ZCMP3 (sense: gga agg aag gag aag aa, anti-sense: ggt cag ggc tgc tta gga ata). Both sense and anti-sense directions were sequenced for correction. In some cases, samples were sent to both the Keck Sequencing Center at Yale Medical School and SeqWright Sequencing Laboratory (Huston) for confirmation. Fresh human brain RNA was used as a normal control for MAP1a. The results were analyzed by DNAstar DNA Analyzing Program. Results: Fragment 1: A length of 560 bp of cDNA was obtained by the amplification of primer set 1. This fragment contains a start codon (tag) in exon 3, in agreement with published information (Schwende H, Fitzke E, Ambs P, Dieter P. Differences in the state of differentiation of THP-1 cells induced by phorbol ester and 1,25-dihydroxyvitamin D3. Journal of Leukocyte Biology, 59:555-61, 1996.). Intron 2 is 210 bp which compares with the published data and the sequence for the MAP1a gene. Fragments 2 and 3 were overlapped and assembled, from which a sequence of 1246 bp was obtained. There were important findings in this fragment: A 4 bp insertion was found which caused a frame-shift mutation that created an immature stop codon (tga) at position 1134-1136. According to these data, the total coding region of this sequence should be 2007 bp, from start codon to stop codon. The deduced amino acid number would be 669. Assuming the average molecular weight of one amino acid residue is 110, then the deduced protein would be 73.59 Kda, which is very close to 80 kda. There were 8 [K/R] [K/R] [K/E] tubulin binding domains in this area, indicating the binding capacity of p80. Comment: The total coding region (start-stop codon) was in near completion, a gap of 483 bp between fragment 1 and fragment 2 remained to be sequenced. 2) The necessary information for designing efficient anti-sense oligos have been collected, i.e., start-stop codons and their flanking areas and binding domains; 3) The use of primer set 1 which included intron 2, exon 2 and exon 3, plus DNase digestion have eliminated the possibility of genomic DNA contamination.

### Example 14

Inhibition of expression of p80 by EM and other drugs which curtail OVCA proliferation. Rationale and Experiments: In order to assess the effects of various classes of chemotherapy on p80-containing OVCA cells and to elucidate the relationship between p80, EM, and OVCA proliferation, EM or tamoxifen or the NADPH-oxidase inhibitor diphenyleneiodonium treatment was carried out. All three agents have been shown to cause apoptosis and lower OVCA cell growth. The treatment was followed with Western blotting to determine the effect of each agent on p80 expression. To control for reduced cell numbers or changes in cell volume as confounding factors, results were calculated on the basis of p80 per mg protein or DNA in the cells. Findings: Expression of p80 was significantly inhibited by EM, tamoxifen and DPI 9. Conclusions: (1) These were experimentally relevant doses since they also significantly decreased cell proliferation in all the cell lines (MTT assay) (data not shown). (2) These results highlighted the significance of studies on p80 with agents, which may specifically interact with MAP's, thereby disabling the proliferative mechanism. The novel E-CC compounds may also be such agents. The three drugs used have different mechanisms of inhibiting cell proliferation. (3) Despite the difference of methods of action of the agents, these studies indicated that the effect on p80 was not simply due to decreased proliferation but represents a true decrease in p80 expression.

### Example 15

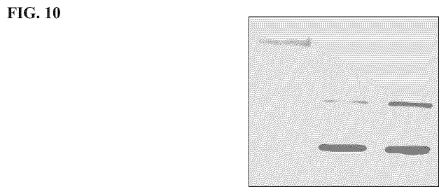

Stimulation of expression of p80 by epidermal growth factor (EGF). Rationale and Experiments: Ovarian cancer cells have been shown to have EGF receptors. To further test the relationship between p80 and OVCA proliferation, OVCA cells were treated with EGF and measured proliferation (MTT assay) and the expression of p80 (Western Blotting). Again, results were calculated on a per mg protein or DNA basis to avoid confounding. Findings: (1) Increasing doses of EGF (10-50 ng/ml) stimulated OVCA proliferation in comparison to untreated controls (data not shown). (2) The expression of p80 was increased significantly by EGF (FIG. 10). Conclusions: (1) The parallelism of p80 expression with OVCA proliferation indicated that p80 may be a key molecule in OVCA proliferation. (2) E-M/ECC's and their roles as specific antimicrotubular agents will be tested. Alternative approaches, such as targeting this molecule by using antisense DNA/RNA to block its expression could be helpful in elucidating whether the expression of this protein is a cause or an effect of OVCA proliferation. Furthermore, the antisense approach may also lead to the development of a novel modality for OVCA therapy targeting p80.

### Example 16

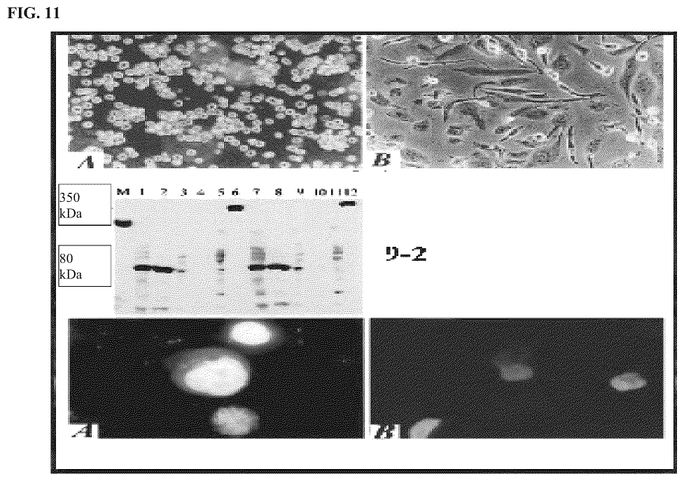

Terminal Differentiation of THP-1 leukemia cells was associated with decreased expression of p80. Rationale: To study the effect on p80 of causing a rapidly proliferating cancer cell line to differentiate. Experimental: A differentiation-inducible cancer model was used, administering phorbol myristic acetate (PMA) to differentiate the promonocytic leukemia cell line, THP-1 into monocytes/macrophages and the expression of p80 was investigated. Cell culture: THP-1 promonocytic leukemia cell lines were obtained from ATCC. Cells were cultured in DMEM medium supplemented with 10% of FBS and antibiotics in 37 C and 5% CO2 incubator. Cells were re-fed every three days. Treatment with phorbol myristic acetate (PMA): PMA (Sigma) was dissolved in absolute ethanol with 10 μg/ml as a stock solution. 10 μl of this stock solution was added to 10 ml culture to make a final concentration of 10ng of PMA/ml. In control cultures, 10 μl absolute ethanol was added instead of PMA solution. Cell morphology was checked daily and photographed. Western blot analysis: After treatment, cells were extracted with lysis buffer. SDS-PAGE and Western blotting were performed as described in the methods, below. Immunofluorescence (IF): suspension-growing THP-1 cells, cells were smeared onto a polylysine-precoated slide and fixed with 4% PFA. For attached THP-1 after PMA treatment, cells were fixed with 4% PFA. Endogenous peroxidase was inactivated by 30% H2O2. Cells were then incubated with monoclonal anti MAP1a overnight. After extensive washing, cells were stained with secondary antibody, FITC labeled horse anti-mouse IgG for Ihr. DAPI was used for counter-staining of nuclei. The slide was studied by a fluorescence microscope for the presence of the P80 and photographed. Results: Before PMA treatment the rapidly dividing THP-1 leukemia cells were round and floating in the culture medium (FIG. 11a Top). Upon PMA treatment, the THP-1 cells began to differentiate to monocytes/macrophages. They attached to the surface and grew processes (FIG. 11b Top), as has previously been reported. Untreated THP-1 cells strongly express p80 on Western analysis. By 5 days of PMA treatment, p80 had disappeared from Western blots (FIG. 11 Middle). Before treatment with PMA, the cells were round with bright fluorescence (FIG. 11-a Bottom); after PMA treatment, the cells grew processes with no IF of P80 (FIG. 11b Bottom). Conclusions: 1) P80 was strongly expressed by proliferating THP-1 cells. 2) This expression was diminished when the differentiation of THP-1 cells was induced by PMA. 3) This further linked P80 with cancer cell proliferation (P80 was not present in proliferating normal cells).

### Example 17

High-Throughput Computational Epitope Selection for P80-Specific Monoclonal Antibody Development.

### Methodology:

P80 Sequence Analysis: High-resolution structural models of P80 variants were generated and computationally dissected using a proprietary suite of algorithms integrating: Physicochemical properties: Surface accessibility, hydrophobicity, flexibility, and electrostatic potential were calculated to pinpoint solvent-exposed, rigid regions favorable for antibody binding. Amino acid residue patterns: Sequence motifs and conserved domains associated with known antibody-binding pockets in other MAPs were identified. HLA-compatibility scoring: Predicted epitopes were scored for binding affinity to diverse HLA class I alleles, enabling personalized immunotherapy potential.

Epitope Prioritization: A composite score integrating the above data prioritized candidate epitopes for high-affinity and specificity against P80.

In vitro Validation: Selected epitopes were synthesized and subjected to biophysical and immunological assays: Surface plasmon resonance (SPR): P80-epitope binding affinity was quantified. Enzyme-linked immunosorbent assay (ELISA): Antibody binding specificity against P80 and other MAPs was evaluated. T-cell activation assays: Epitope presentation by HLA molecules and subsequent T-cell activation potential for immunotherapy was confirmed.

An alternative method is through commercial outlets that use proprietary algorithms to identify likely epitopes, synthesize candidate sequences and raise the monoclonal antibodies to them through the use of hybridoma technology, with standard testing for specificity and sensitivity.

### THERAPEUTIC COMPOSITIONS

Pharmaceutical or therapeutic compositions of the present disclosure are disclosed by way of example and not limitation. Those skilled in the art will be versed in making modifications of substitutions of various components, ingredients, dosages and treatment regimens.

The administration to a subject in need of the therapeutic pharmaceutical compositions of the present disclosure may be an intravenous infusion, oral ingestion, inhalation, intramuscular injection, subcutaneous injection, intravaginal application, and dermal and ocular penetration.

Pharmaceutical compositions for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an artemisinin-related compound as an active ingredient. An artemisinin-related compound may also be administered as a bolus, electuary or paste.

### NANOPARTICLES IN THERAPEUTIC COMPOSITIONS

Nanoparticles include endosomes, lipid rafts and low nanometer-sized artificial containers, known in the art, that carry cargos which may affect the targeted intracellular proteins or other vital intracellular molecules or structures. The cargo may be pharmaceuticals, natural interfering agents, synthetic molecules or radioactive materials. The agents may be auto fluorescent, x-ray or ultrasound opaque, etc. and be imaged directly at surgery or registered by imaging equipment.

The instant disclosure discloses targeting p80 by linking protein-specific monoclonal antibodies or binding small molecules to nanoparticles, or structures, small enough to bind to or enter living cells that express p80 proteins. The nanoparticles or other vesicles, including lipid rafts, that carry cargos such as radioactive agents, chemotherapeutic drugs, or other agents that sabotage cell function, thereby inactivating or destroying these cells. In this case the antibodies, nanoparticles or small molecules act as drug delivery systems. In addition, once they have been linked to the p80 it is not necessary that p80 be affected by the monoclonal antibodies, nanoparticles or small molecules, themselves. Rather, the attached or cargo agents may be the treatment. The small molecules, by binding to the target proteins may themselves inactivate them. The affected cells are then disposed of by the usual cellular mechanisms including apoptosis, autophagy, etc.

The complexed nanoparticles may be of a size ranging from 0.1 μm and 1.0 μm. The shape of the nanoparticle may be a sphere, cuboidal or elongated depending on the desired permeability characteristics of a target cell. Nanoparticles also have surface functionality. For instance, to bind poly-ethylene-glycol to increase circulation time to prolong the therapeutic effect of the complexed cargo on the nanoparticle.

The ability to deliver multiple copies of a radiotherapeutic to a single receptor site is perhaps the most useful property that can be combined using a nanoconjugate, which consists of a nanoparticle, a linking agent, and an antibody or peptide. Other useful properties include tuning the biodistribution by altering the nanoparticle's surface. When compared to currently approved targeted radiotherapies, the cytotoxicity of nanoparticle-based medicines will be higher when many radioactive atoms are delivered to each receptor. Modular surface modification enables the nanoparticle system to be biodistributed specifically to enhance accumulation at the tumor site.

Antibody labeling of gold-coated lanthanide phosphate nanoparticles can produce promising theragnostic anti-cancer nanoconjugates. The intermediate energy beta emitted in the decay can be quite effective in treating metastatic disease. The 208 keV gamma-ray from 177Lu decay (11%) can be employed for SPECT imaging of the radiotherapeutic drug. Each nanoparticle would contain three radioactive atoms on average if 20 mCi of activity were used in the synthesis.

Protocols for conjugating p80 binding agents to nanoparticles, or therapeutic agents to nanoparticles, will vary based on the selected nanoparticles. Commercial kits are also available to facilitate this complex.

By way of example and not limitation, the cargo on the nanoparticle may be a therapeutic pharmaceutical agent selected from the group consisting of: methotrexate, amsacrine, azacytidine, bleomycin, busulfan, capecitabine, carboplatin, carmustine, chlorambucil, cisplatin, cladribine, cyclophosphamide, cytarabine, dactinomycin, daunombicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, vinorelbine, aminoglutethimide, anastrozole, asparaginase, bcg, bicalutamide, buserelin, campothecin, clodronate, colchicine, cyproterone, dacarbazine, dienestrol, diethylstilbestrol, estradiol, exemestane, filgrastim, fludrocortisone, fluoxymesterone, flutamide, genistein, goserelin, hydroxyurea, imatinib, interferon, ironotecan, letrozole, leucovorin, leuprolide, levamisole, medroxyprogesterone, megestrol, mesna, nilutamide, nocodazole, octreotide, pamidronate, porfimer, raltitrexed, rituximab, suramin, tamoxifen, temozolomide, testosterone, titanocene dichloride, trastuzumab, tretinoin, vindesine, HERCEPTIN® and other antibody therapeutics, and an anti-sense or RNAi agent against one or more genes promoting the progression of the cancer.

Multiple therapeutic agents and multiple p80-binding agents may be complexed to a nanoparticle, determined by the condition being treated the severity, aggressiveness of the progression of the proliferative condition and other clinical variables. It will be obvious to those skilled in the art to vary the complexed components to increase specificity and therapeutic value of a nanoparticle treatment.

### EQUIVALENTS

Those skilled in the art will recognize or be able to ascertain using no more than routine experimentation, numerous equivalents to the specific method and reagents described herein, including alternatives, variants, additions, deletions, modifications, and substitutions. Cancer or proliferative diseases in non-human species expressing p80 are targets for the same agents as described above.

Subjects of the disclosure may be any human, including pregnant women. The instant disclosure is also applicable to domesticated animals, or non-domesticated animals. The uses of the present disclosure may be used in human medicine and veterinary medicine.

What is claimed is:

1. A method of screening, diagnosis, clinical staging or assessment of treatment for cancer or proliferative disease in a subject in need thereof, the method comprising:
(a) obtaining a tissue/cellular or biological fluid sample from the subject;
(b) diluting the tissue sample in a buffer solution;
(c) contacting the tissue sample with a monospecific antibody that recognizes p80 protein;
(d) detecting complex formation or binding of the antibody to p80 protein;
(e) wherein complex formation or binding is detected with an ELISA sandwich assay,
wherein the antibody that recognizes p80 protein is a capturing antibody bound to a substrate, wherein the capturing antibody bound to the substrate is contacted with the sample, and wherein complex formation is measured by the binding of a tagged secondary antibody that recognizes p80, wherein the antibody comprises a monospecific antibody that binds to a peptide epitope of p80 that ranges in length from 4 to 20 amino acids of the amino acid sequence described by SEQ ID NO:1.

2. The method of claim 1, wherein the antibody is a polyclonal antibody(s).

3. The method of claim 1, wherein the antibody comprises a polyclonal monospecific antibody for p80 and tubulin.

4. The method of claim 1, wherein the antibody comprises a monospecific antibody that binds to a C-terminal domain segment of p80 protein.

5. The method of claim 1, wherein said disease comprises carcinomas, sarcomas, lymphomas and other malignant, pre-malignant, in situ, metastatic or other neoplasms, including leukemias, other blood cancers, fibromas, gliomas, or active proliferative diseases.

6. The method of claim 1, wherein said tissue sample is selected from exfoliated cells, fragmented cells, tissue scrapings including pap smear, fluids/blood, urine, gut content, endoscopy, aspiration, biopsy, or endoscopic tool.

7. The method of claim 1, wherein said subject is human or animal.

8. The method of claim 7, wherein said human is female or male, of any age, including the unborn.

9. The method of claim 1, further comprising testing for the effects of surgical or medical treatment of said lesions.

10. A method of using antibodies to identify the presence of lesions in cancer or proliferative disease, the method comprising:
(a) obtaining a tissue sample from the subject;
(b) diluting the tissue sample in a buffer solution that makes the sample prepared for testing or prepares the sample for suitable study, such as wax impregnation and sectioning by a microtome, or other method of tissue preparation for histological and marker antibody study;
(c) contacting the tissue sample with separate monovalent antibodies or a bivalent antibody that recognizes p80 and tubulin protein;
(d) detecting complex formation or binding of the antibody to p80 and/or tubulin protein; and
(e) wherein complex formation or binding is detected with an ELISA sandwich assay;
wherein the antibody that recognizes the protein(s) is a capturing antibody bound to a substrate, wherein the capturing antibody bound to the substrate is contacted with the sample, and wherein complex formation is measured by the binding of a tagged secondary antibody that recognizes p80, wherein the antibody comprises a monospecific antibody that binds to a peptide epitope of p80/tubulin that ranges in length from 4 to 20 amino acids of the amino acid sequence of p80 described by SEQ ID NO:1.

11. The method of claim 10, wherein said method is performed in a clinical setting.

12. The method of claim 10, wherein said method detects the presence of p80 protein in the substantial absence of cross-reactivity with other proteins.

13. A composition comprising the antibody of claim 12.

14. A method for diagnosis and treatment of a gynecologic disease comprising detecting an amount of p80 polypeptide consisting of SEQ ID NO: 1 in a human subject blood, serum or plasma sample from a human patient suspected of suffering from said gynecologic disease wherein the amount of p80 polypeptide is increased as compared to a control; and treating the subject to target the cancer.

15. The method of claim 1 wherein the amount of the p80 polypeptide is determined by contacting the human or animal sample with one or more antibodies or antigen binding fragments specific for full length human p80 under conditions suitable for polypeptide/antibody complexes to form and detecting the polypeptide/antibody complexes.

16. The method of claim 14, wherein detecting the amount of polypeptide is performed by an immunoassay selected from the group consisting of an enzyme linked immunosorbent assay (ELISA), western blot, immunofluorescence assay (IFA), radio immunoassay, hemagglutinin assay, fluorescence polarization immunoassay, microtiter plate assays, reversible flow chromatographic binding assay, and immunohistochemistry assay.

17. The method of claim 15, wherein the one or more antibodies or antigen binding fragments are detectably labeled.

18. The method of claim 15, wherein the one or more antibodies or antigen binding fragments are immobilized to a solid support.

19. The method of claim 15, wherein the one or more antibodies or antigen binding fragments are monoclonal antibodies, single chain antibodies, polyclonal antibodies, or antigen binding fragments (Fab fragments).

20. A method for detecting a p80 polypeptide defined by SEQ ID NO: 1 or any antigenic fragments thereof in a test sample, comprising:
(a) obtaining a test sample from a patient; and
(b) detecting whether p80 is present in the test sample by contacting the test sample with an anti-p80 antibody and detecting binding between p80 and the anti-p80 antibody.

21. The method of claim 20, wherein the step of detecting binding between p80 and the anti-p80 antibody comprises:
i. providing a reaction vessel, coated with a capture antibody onto its surface;
ii. adding a test sample comprising the target antigen into the reaction vessel to facilitate binding between the bound antibody and the target antigen;
iii. washing the solid substrate in the reaction vessel to remove any excess, target antigen not bound to the solid substrate;
iv. introducing the detection antibody into the reaction vessel to facilitate binding between the target molecules bound to the capture antibody and the detection antibody;
v. washing the solid substrate in the reaction vessel to remove any excess detection antibody not bound to the target molecule; and
vi. quantifying the amount of sandwiched target antigen by the presence of aggregated detection antibody-target antigen-capture antibody based on measurement of optical density.


SEQUENCE LISTING (1 sequence)

SEQ ID NO: 1 - protein, 1825 aa, Homo sapiens
  VAR_SEQ 802
MDGVAEFSEYVSETVDVPSPFDLLEPPTSGGFLKLSKPCCYIFPGGRGDSALFAVNGFNILVDGGSDRKSCFWKLVRHLDRIDSVLLTHIGADNLPGINGLLQRKVAELEEEQSQGSSSYSDWVKNLISPELGVVFFNVPEKLRLPDASRKAKRSIEEACLTLQHLNRLGIQAEPLYRVVSNTIEPLTLFHKMGVGRLDMYVLNPVKDSKEMQFLMQKWAGNSKAKTGIVLPNGKEAEISVPYLTSITALVVWLPANPTEKIVRVLFPGNAPQNKILEGLEKLRHLDFLRYPVATQKDLASGAVPTNLKPSKIKQRADSKESLKATTKTAVSKLAKREEVVEEGAKEARSELAKELAKTEKKAKESSEKPPEKPAKPERVKTESSEALKAEKRKLIKDKVGKKHLKEKISKLEEKKDKEKKEIKKERKELKKDEGRKEEKKDAKKEEKRKDTKPELKKISKPDLKPFTPEVRKTLYKAKVPGRVKIDRSRAIRGEKELSSEPQTPPAQKGTVPLPTISGHRELVLSSPEDLTQDFEEMKREERALLAEQRDTGLGDKPFPLDTAEEGPPSTAIQGTPPSVPGLGQEEHVMKEKELVPEVPEEQGSKDRGLDSGAETEEEKDTWEEKKQREAERLPDRTEAREESEPEVKEDVIEKAELEEMEEVHPSDEEEEDATKAEGFYQKHMQEPLKVTPRSREAFGGRELGLQGKAPEKETSLFLSSLTTPAGATEHVSYIQDETIPGYSETEQTISDEEIHDEPEERPAPPRFHTSTYDLPGPEGAGPFEASQPADSAVPATSGKVXGTPETELTYPTNIVAAPLAEEEHVSSATSITECDKLSSFATSVAEDQSVASLTAPQTEETGKSSLLLDTVTSIPSSRTEATQGLDYVPSAGTISPTSSLEEDKGFKSPPCEDFSVTGESEKRGEIIGKGLSGERAVEEEEEETANVEMSEKLCSQYGTPVFSAPGHALHPGEPALGEAEERCLSPDDSTVKMASPPPSGPPSATHTPFHQSPVEEKSEPQDFQEADSWGDTKRTPGVGKEDAAEETVKPGPEEGTLEKEEKVPPPRSPQAQEAPVNIDEGLTGCTIQLLPAQDKAIVFEIMEAGEPTGPILGAEALPGGLRTLPQEPGKPQKDEVLRYPDRSLSPEDAESLSVLSVPSPDTANQEPTPKSPCGLTEQYLHKDRWPEVSPEDTQSLSLSEESPSKETSLDVSSKQLSPESLGTLQFGELNLGKEEMGHLMQAEDTSHHTAPMSVPEPHAATASPPTDGTTRYSAQTDITDDSLDRKSPASSFSHSTPSGNGKYLPGAITSPDEHILTPDSSFSKSPESLPGPALEDIAIKWEDKVPGLKDRTSEQKKEPEPKDEVLQQKDKTLEHKEVVEPKDTAIYQKDEALHVKNEAVKQQDKALEQKGRDLEQKDTALEQKDKALEPKDKDLEEKDKALEQKDKIPEEKDKALEQKDTALEQKDKALEPKDKDLEQKDRVLEQKEKIPEEKDKALDQKVRSVEHKAPEDTVAEMKDRDLEQTDKAPEQKHQAQEQKDKVSEKKDQALEQKYWALGQKDEALEQNIQALEENHQTQEQESLVQEDKTRKPKMLEEKSPEKVKAMEEKLEALLEKTKALGLEESLVQEGRAREQEEKYWRGQDVVQEWQETSPTREEPAGEQKELAPAWEDTSPEQDNRYWRGREDVALEQDTYWRELSCERKVWFPHELDGQGARPHYTEERESTFLDEGPDDEQEVPLREHATRSPWASDFKDFQESSPQKGLEVERWLAESPVGLPPEEEDKLTRSPFEIISPPASPPEMVGQRVPSAPGQESPIPDPKL